(19) United States
(12) Patent Application Publication
(10) Pub. No.: US 2015/0335624 A1
(43) Pub. Date: Nov. 26, 2015

(54) COMPOSITIONS AND METHODS FOR USING HUPERZINE AND ANALOGS THEREOF

(71) Applicant: INSERO HEALTH INC., Miami, FL (US)

(72) Inventors: Stephen D. COLLINS, Lake Forest, IL (US); Stephen P. WANASKI, Chicago, IL (US); Holli A. CARLSON, Arlington Heights, IL (US); David KOLB, Miami, FL (US)

(21) Appl. No.: 14/655,899

(22) PCT Filed: Jan. 6, 2014
(86) PCT No.: PCT/US14/10384

§ 371 Date: Jun. 26, 2015

Related U.S. Application Data
(60) Provisional application No. 61/749,199, filed on Jan. 4, 2013.

(51) Int. Cl. A61K 31/439

(52) U.S. Cl. CPC A61K 31/439

(57) ABSTRACT

A method of treating a seizure disorder is described wherein an acetylcholinesterase(AChE) inhibitor is administered to a subject having a seizure disorder and an increased risk of a cardiac event from the seizure disorder, wherein the AChE inhibitor decreases the risk of such cardiac event. Further described are methods of decreasing the risk of a cardiac event in subjects with or without a seizure disorder by administering a therapeutically effective amount of an AChE inhibitor. Methods of treating kidney disease and reducing an elevated CRP level by administering an AChE inhibitor are also described.

### BRIEF SUMMARY OF THE INVENTION

In an embodiment, a method of treating a seizure disorder includes administering to a subject in need of such treatment a therapeutically effective amount of an acetylcholinesterase (AChE) inhibitor, wherein the subject has an increased risk of a cardiac event from such a seizure disorder, and wherein the AChE inhibitor decreases the risk of such cardiac event.

In an embodiment, a method of decreasing the risk of a cardiac event in a subject with a seizure disorder includes administering to the subject in need of such treatment a therapeutically effective amount of an AChE inhibitor, wherein the subject has an increased risk of a cardiac event from the seizure disorder, and wherein the AChE inhibitor decreases the risk of such cardiac event.

In an embodiment, a method of treating a seizure disorder includes administering to a subject in need of such treatment a therapeutically effective amount of an AChE inhibitor, wherein the AChE inhibitor does not prolong said subject's QTc interval and wherein the seizure disorder is treated.

In an embodiment, a method of decreasing the risk of a cardiac event in a subject without a seizure disorder includes administering to the subject in need of such treatment a therapeutically effective amount of an AChE inhibitor, wherein said subject has an increased risk of a cardiac event, and wherein the AChE inhibitor decreases the risk of such cardiac event.

In an embodiment, a method for treating a kidney disease includes administering to a subject in need of such treatment a therapeutically effective amount of an AChE inhibitor, wherein the kidney disease is treated.

In an embodiment, a method of reducing an elevated CRP level in a subject includes administering to a subject in need of such treatment a therapeutically effective amount of an AChE inhibitor, wherein the CRP level is reduced.

### DESCRIPTION OF DRAWINGS

FIG. 1 is a graph showing a change in peak plasma levels of IL-6 in subjects according to an embodiment of the invention.

FIG. 2 shows two graphs showing a decrease in plasma creatinine in subjects according to the embodiment of FIG. 1.

FIG. 3 shows two graphs showing an increase in glomerular filtration rate in subjects according to the embodiment of FIG. 1.

FIG. 4 is a graph showing a decrease in CRP in subjects according to the embodiment of FIG. 1.

FIG. 5 is a graph showing the hourly heart rate trends for the 8 patients for the full recording period of the study described in Example 1.

FIG. 6 is a graph showing the hourly heart rate trends for the 8 patients for the initial 12 hours of recording of the study described in Example 1,

FIG. 7 is a graph showing the hourly heart rate variability trends for the 8 patients for the full recording period of the study described in Example 1.

FIG. 8 is a graph showing the hourly heart rate variability trends for the 8 patients for the initial 12 hours of recording of the study described in Example 1.

FIG. 9 is a graph showing the hourly trends in T-wave alternans for the 8 patients for the full recording period of the study described in Example 1.

FIG. 10 is a graph showing the hourly trends in QT interval length for the 8 patients for the full recording period of the study described in Example 1.

FIG. 11 is a graph showing the hourly trends in QTc interval length for the 8 patients for the full recording; period of the study described in Example 1 as corrected using Bazett's formula.

FIG. 12 is a graph showing the hourly trends in ventricular premature beat counts for the 8 patients for the full recording period of the study described in Example 1.

FIG. 13 is a graph showing the hourly ventricular tachycardia count trend for the 8 patients for the full recording period of the study described in Example 1.

### DETAILED DESCRIPTION

Before the present compositions and methods are described, it is to be understood that this invention is not limited to the particular processes, compositions, or methodologies described, as these may vary. It is to be also understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

Optical isomers-diastereomers-geometric isomers-tautomers. Compounds described herein may contain an asymmetric center and may thus exist as enantiomers. Where the compounds according to the invention possess two or more asymmetric centers, they may additionally exist as diastereomers. The present invention includes all possible stereoisomers as substantially pure resolved enantiomers, racemic mixtures thereof as well as mixtures of diastereomers. The formulas are shown without a definitive stereochemistry at certain positions. The present invention includes all stereoisomers of such formulas and pharmaceutically acceptable salts and solvates thereof Diastereoisomeric pairs of enantiomers may be separated by, for example, fractional crystallization from a suitable solvent, and the pair of enantiomers thus obtained may be separated into individual stereoisomers by conventional means, for example, by use of an optically active acid or base or a resolving agent or on a chiral HPLC column. Further, any enantiomer or diastereomer of a compound of the general formula may be obtained by stereospecific using optically pure starting materials or reagents of known configuration.

It must be noted that as used herein and in the appended claims, the singular firms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used, Therefore, about 50% means in the range of 45%-55%.

“Administering” when used in conjunction with a therapeutic means to administer a therapeutic agent into or onto a target tissue or to administer a therapeutic to a subject whereby the therapeutic agent positively impacts the tissue to which it is targeted. Administering may be done by the actual subject being treated or a health care professional.

The terms “individual”, “host”, “subject”, “patient”, and “animal” as used interchangeably herein include, but are not limited to, humans and non-human vertebrates such as wild, domestic and farm animals.

The term “improves” as used herein, is used to convey that the present invention changes the appearance, form, characteristics, physiological, and/or the physical attributes of the tissue and/or organ to which it is being provided, applied or administered.

The term “inhibiting” includes the administration of a compound of the present invention to prevent the onset of the symptoms, alleviating the symptoms, reducing the symptoms, delaying or decreasing the progression of the disease or its symptom, or eliminating or ameliorating the disease, condition or disorder.

By “pharmaceutically acceptable”, it is meant the carrier, diluent, excipient, or counter ion must be compatible with the other ingredients of the formulation and not deleterious to the recipient thereof.

Pharmaceutically acceptable salts as anionic counter ions include, but are not limited to, acetate, bromide, camsylate, chloride, formate, fumarate, maleate, mesylate, nitrate, oxalate, phosphate, sulfate, tartrate, thiocyanate, tosylate, adipate, caprate, caproate, caprylate, dodecylsulfate, glutarate, laurate, oleate, palmitate, sebacate, stearate, undecylenate, and combinations thereof. Pharmaceutically acceptable salts as cationic counter ions include, but are not limited to, ammonium, arginine, diethylamine, ethylenediamine, piperazine, and combinations thereof. Pharmaceutically acceptable salts include, but are not limited to, chloride, bromide, nitrate, sulfate, tosylate, phosphate, tartrate, or maleate. Pharmaceutically acceptable compounds include hydrates thereof.

As used herein, the term the “therapeutic” means an agent utilized to treat, combat, ameliorate, prevent or improve an unwanted condition or disease of a subject.

A “therapeutically effective amount” or “effective amount” of a composition is a predetermined amount calculated to achieve the desired effect such as to treat, combat, ameliorate, prevent or improve an unwanted condition or disease of a subject. The activity contemplated by the present methods includes both medical therapeutic and/or prophylactic treatment, as appropriate. The specific dose of a compound administered according to this invention to obtain therapeutic and/or prophylactic effects will be determined by the particular circumstances surrounding the case, including, for example, the compound administered, the route of administration, and the condition being treated. The compounds are effective over a wide dosage range and, for example, dosages per day will normally fall within the range of from 0.001 to 20 mg/kg, more usually in the range of from 0.01 mg/kg to 1 mg/kg. However, it will be understood that the effective amount administered will be determined by the physician/clinician in the light of the relevant circumstances including the conditions to be treated, the choice of compound to be administered, and the chosen route of administration, and therefore the above dosage ranges are not intended to limit the scope of the invention in any way. A therapeutically effective am with of compound of this invention typically an amount such that when it is administered in a physiologically tolerable excipient composition, it is sufficient to achieve an effective systemic concentration or local concentration in the e tissue.

The terms “treat ”, “treated”, “treating” as used herein refer to both therapeutic treatment and preventative measures, wherein the object is to prevent or slow down an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The terms “carrier”, “excipient”, “diluent”, and “adjuvant” may be used interchangeably and refer to a composition with which the therapeutic agent is administered. Such carriers may be sterile liquids such as, for example, water and oils, including those of petroleum, animal, vegetable or synthetics origin. Saline solution, aqueous dextrose and glycerol solution may also be employed as liquid carriers. Suitable pharmaceutical excipients include, but are not limited to, glucose, starch, lactose, sucrose, gelatin, malt, rice, flour, chalk, sodium chloride, dried skim milk., glycerol, propylene, glycol, water, and ethanol. The composition, if desired, may contain minor amounts of wetting or emulsifying agents, or pH buffering agents. These compositions may take a form of solutions, suspensions, emulsions, powders, sustained-release formulations, and the like.

The term “alkyl,” as used herein, refers to a branched or unbranched saturated hydrocarbon group of 1 to 24 carbon atoms, such as, without limitation, methyl, ethyl, n-propyl, isopropyl, n-butyl, isobutyl, Cert-butyl, pentyl, hexyl, heptyl, octyl, decyl and like. Preferred alkyl groups herein contain 1 to 6 carbon atoms. Alkyl groups may be optionally substituted with one to three groups chosen from halo, amino, methoxy, ethoxy, hydroxyl, methylthio, methylsulfonyl, nitro, aryl, heterocyclyl and heteroaryl.

The term “alkenyl,” as used herein, refers to a branched or unbranched hydrocarbon group of 2 to 24 carbon atoms containing at least one unsaturated bond, such as, without limitation, vinyl, propenyl, butenyl, pentenyl, hexenyl, heptenyl, octenyl, decenyl, and the like. Preferred alkenyl groups herein contain 2 to 6 carbon atoms. Alkenyl groups may be optionally substituted with one to three groups chosen from halo, amino, methoxy, ethoxy, hydroxyl, methylthio, methylsulfonyl, nitro, aryl, heterocyclyl and heteroaryl.

The term “cycloalkyl” refers to ring-containing alkyl radicals of 3 to 14 carbon atoms. Examples include cyclohexyl, cyclopentyl, cyclopropyl, cyclopropylmethyl and norbornyl. Cycloalkyl groups may be optionally substituted with one to three groups chosen from halo, amino, methoxy, ethoxy, hydroxyl, methylthio, methylsulfonyl, nitro, aryl, heterocyclyl and heteroaryl.

The term “aryl” or “Ar” employed alone or in combination with other terms, means, unless otherwise stated, a carbocyclic aromatic group containing one or more rings (typically one, two or three rings). Multiple rings may be attached together in a pendent manner, such as a biphenyl, or may be fused, such as naphthalene. Examples include, but are not limited to, phenyl, anthracyl and naphthyl. Preferred are phenyl (Ph) and naphthyl, most preferred is phenyl. Aryl groups may be optionally substituted with one to three groups chosen from halo, amino, methoxy, ethoxy, hydroxyl, methylthio, methylsulfonyl, nitro, aryl, heterocyclyl and heteroaryl.

The term “heterocycle” “heterocyclyl” or “heterocyclic” by itself or as part of another substituent means, unless otherwise stated, an unsubstituted or substituted, stable, mono- or multicyclic heterocyclic ring system consisting of carbon atoms and at least one heteroatom including, but not limited to, N, O, and S, and wherein the nitrogen and sulfur heteroatoms may be optionally oxidized, and the nitrogen atom may be optionally quaternized. The heterocycle may be attached to the compound of which it is a component, unless otherwise stated, at any heteroatom or carbon atom in the heterocycle that affords a stable structure. Heterocyclic groups may be optionally substituted with one to three groups chosen from halo, amino, methoxy, ethoxy, hydroxyl, methylthio, methylsulfonyl, nitro, aryl, heterocyclyl and heteroaryl.

Examples of non-aromatic heterocycles include monocyclic groups such as: aziridinyl, oxiranyl, thiiranyl, azetidinyl, oxetanyl, thietanyl, pyrrolidinyl, pyrrolinyl, imidazolinyl, pyrazolidinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, 1,4-dihydropyridinyl, piperazinyl, morpholinyl, thiomorpholinyl, pyranyl, 2,3-dihydropyranyl, tetrahydropyranyl, 1,4-dioxanyl, 1,3-dioxanyl, homopiperazinyl, homopiperidinyl, 1,3-dioxepinyl, 4,7-dihydro-1,3-dioxepinyl and hexamethyleneoxide.

The term “heteroaryl” or “heteroaromatic” refers to a heterocycle having aromatic character. A monocyclic heteroaryl group is preferably a 5-, 6-, or 7-membered ring, examples of which are pyrrolyl, furyl, thienyl, pyridyl, pyrimidinyl and pyrazinyl. A polycyclic heteroaryl may comprise multiple aromatic rings or may include one or more partially saturated rings. Heteroaryl groups may be optionally substituted with one to three groups chosen from halo, amino, methoxy, ethoxy, hydroxyl, methylthio, methylsulfonyl, nitro, aryl, heterocyclyl and heteroaryl.

Examples of monocyclic heteroaryl groups include, for example, six-membered monocyclic aromatic rings such as, for example, pyridyl, pyrazinyl, pyrimidinyl and pyridazinyl; and five-membered monocyclic aromatic rings such as, for example, thienyl, furyl, pyrrolyl, imidazolyl, thiazolyl, oxazolyl, pyrazolyl, isothiazolyl, 1,2,3-triazolyl, 1,2,4-triazolyl, 1,3,4 triazolyl, tetrazolyl, 1,2,3-thiadiazoloyl, 1,2,3-oxadiazolyl, 1,3,4-thiadiazolyl and 1,3,4-oxadiazolyl.

Examples of polycyclic heteroaryl groups containing a partially saturated ring include tetrahydroquinolyl and 2,3-dihydrobenzofuryl.

Examples of polycyclic heteroaryls include indolyl, indolinyl, quinolyl, tetrahydroquinolyl, isoquinolyl, 1,2,3,4-tetrahydroisoquinolyl, cinnolinyl, quinoxalinyl, quinazolinyl, phthalazinyl, 1, 8 -naphthyridinyl, 1,4-benzodioxanyl, chromene-2-one-yl (coumarinyl), dihydrocoumarin, chromene-4-one-yl benzofuryl, 1,5-naphthyridinyl, 2,3-dihydrobenzofuryl, 1,2-benzisoxazolyl, benzothienyl, benzoxazolyl, benzothiazolyl, purinyl, benzimidazolyl, benzotriazolyl, thioxanthinyl, benzazepinyl, benzodiazepinyl, carbazolyl, carbolinyl, acridinyl, pyrrolizidinyl and quinolizidinyl.

The term “substituted” refers to a molecular group that replaces a hydrogen in a compound and may include, but are not limited to, trifluoromethyl, nitro, cyano, C₁-C₂₀ alkyl, aromatic or aryl, halide (F, Cl, Br, I), C₁-C₂₀ alkyl ether, benzyl halide, benzyl ether, aromatic or aryl ether, hydroxy, alkoxy, amino, alkylamino (—NHR′), dialkylamino (—NR″R″) or other groups which do not interfere with the formation of the diaryl alkylphosphonate.

As used herein, the term “seizure disorder” means any condition in which one or more seizures is a symptom. As used herein, a seizure may be due to unusual electrical activity in the brain or may be a non-epileptic seizure, which is not accompanied by abnormal electrical activity in the brain. A seizure may be caused by, for example, but not limited to, psychological issues, psychological stress, trauma, hypoglycemia, low blood sodium, fever, alcohol use, or drug use or unknown causes. Types of seizures and seizure disorders include but are not limited to, epilepsy, generalized seizures, primary generalized seizures, absence seizures, myoclonic seizures, partial seizures, and complex partial seizures with or without generalization. In some embodiments, the seizure disorder is epilepsy.

As used herein, the term “epilepsy” refers to a disorder of the brain characterized by an enduring predisposition to generate epileptic seizures and by the neurobiologic, cognitive, psychological, and social consequences of this condition. An epileptic seizure is a transient occurrence of signs and/or symptoms due to abnormal excessive or synchronous neuronal activity in the brain.

As used herein, the term “Dravet Syndrome” (also called “Severe Myoclonic Epilepsy of infancy” or SMEI) refers to a form of intractable epilepsy that begins in infancy. In Dravet Syndrome initial seizures are most often prolonged events and in the second year of life other seizure types typically begin to emerge. Generalized Epilepsy with Febrile Seizures Plus (GEFS+)is one of the Dravet Spectrum Disorders, which is one of a group of related seizure disorders with a similar genetic disorder. Individuals with Dravet Syndrome and related disorders (such as GEFS+) face a higher incidence of sudden unexplained death in epilepsy and have other associated conditions.

The term “sudden unexplained death in epilepsy” refers to the death of a person with epilepsy, wherein death results from unexplained respiratory failure or cardiac arrest after seizures. The exact initiation of sudden unexplained death in epilepsy is unknown in most people. Often irregular rhythms of the heart, such as ventricular tachycardias are end-stage events in people who die of sudden unexplained death in epilepsy. Abnormal cardiac rhythms that predispose a person to fatal ventricular arrhythmias include abnormal T-waves. A person with repeated, convulsive seizures is at greater risk for abnormal T-wave patterns, dispose the person to fatal outcomes. Examples of individuals susceptible to sudden unexplained death in epilepsy include, for example, individuals with Dravet Syndrome, individuals with refractory complex partial seizures with secondary generalization, individuals with high frequency of generalized seizures, and individuals with abnormal electrocardiograms, in particular abnormal T-wave alternans (TWAs).

As used herein, the term “renal failure” means a disease state or condition wherein the renal tissues fail to perform their nominal functions. Renal failure includes chronic and acute renal failure or dysfunction. Acute renal failure is broadly defined as a rapid deterioration in renal function sufficient to result in accumulation of nitrogenous wastes in the body. The causes of such deterioration include renal hypoperfusion, obstructive uropathy, and intrinsic renal disease such as acute glomerulonephritis. Chronic renal failure is usually caused by renal injuries of a more sustained nature which often lead to progressive destruction of nephron mass. Glomerulonephritis, tubulointerstitial diseases, diabetic nephropathy and nephrosclerosis are among the most common causes of chronic renal failure. Chronic renal failure can be defined as a progressive, permanent and significant reduction in glomerular filtration rate (GER) due to a significant and continuing loss of nephrons. The clinical syndrome that results from profound loss of renal function is called uremia.

Renal failure can be divided into several stages starting from mild form followed by moderate and severe forms and processing to so-called end stage renal disease. These stages can be identified in a conventional way, e.g., by determining the creatinine clearance values for which well-defined ranges are assigned to the different stages of renal insufficiency.

Diagnostic signs of renal failure include lower than creatinine clearance; lower than normal free water clearance; higher than normal blood urea and/or nitrogen and/or potassium and/or creatinine levels; altered activity of kidney enzymes such as gamma glutamyl synthetase; altered urine osmolarity or volume; elevated levels microalbuminuria or macroalbuminuria; glomerular and arteriolar lesions; tubular dilation; hyperphosphatemia; or need for dialysis.

The inhibition of the renal failure can be evaluated by measuring these parameters in mammals by methods well known in the art, e.g., by measuring creatinine clearance.

The term “diabetic neuropathy” relates to any form of diabetic neuropathy, or to one or more symptom(s) or disorder(s) accompanying or caused by diabetic neuropathy, or complications of diabetes affecting nerves. In diabetic polyneuropathy, many nerves are simultaneously affected. In focal mononeuropathy, the disease affects a single nerve, such as the oculomotor or abducens cranial nerve. The disorder is called multiple mononeuropathy when two or more nerves are affected in separate areas.

Interleukin 6 (IL-6) is an interleukin that acts as both a pro-inflammatory and anti-inflammatory cytokine. In humans, it is encoded by the IL6 gene. An increase in IL-6 has positive clinical significance. Controlled increases in IL-6 are neuroprotective and anticonvulsant. IL-6 increases where antiepileptic drugs (AEDs), such as carabersat (CRB) and valproic acid (VPA) are successful. Clinical and Experimental Medicine, Vol. 1, No. 3 (2001), 133-166. IL-6 increases post-successful surgical resection while other inflammatory markers dropped. J. Neuroimmunol. 2012 Oct. 29, pii. IL-6 is neuroprotective in the face of NMDA excitotoxicity. Journal of Immunology, 1999, 163: 3963-68. IL-6 is anticonvulsant, reducing seizure frequency, latency and duration. Barin and Development, 29 (2007) 644-48. IL-6-/- mice exhibit significantly higher susceptibility to seizure. Pharmacology, Biochemistry and Behavior 77 (2004) 761-66. IL-6 protected animals from chemically induced convulsing seizures. Neuropsychopharmacology, 2008 Aug. 33(9): 2237-50. Each of which is incorporated in their entirety.

Not to be bound to any theory, the increases in IL-6 may be mediated through activation of Nrf2. Nrf2 is the primary cellular defense against cytotoxic effects of oxidative stress. N. Eng. J. Med 367 (12): 1098-1107. The ability for excitatory agent to induce Nrf2 translocation was significantly decreased by IL-6-/- mice. Free Radic. Biol. Med. 2010 Apr. 1; 52(7); 1159-74, Nrf2 is a potent activator of IL-6 gene transcription. J. Bol. Chem. 2011 Feb 11; 286(6): 4493-99. Each of which is incorporated in their entirety.

C-reactive protein (CRP) is protein found in the blood, the levels of which rise in response to inflammation (i.e. CRP is an acute-phase protein). Elevations of CRP in the absence of clinically significant inflammation can occur in a number of diseases, including renal failure and epilepsy. CRP level is an independent risk factor for atherosclerotic disease. Subjects with high CRP concentrations are more likely to develop stroke, myocardial infarction, and severe peripheral vascular disease.

Elevated levels of CRP also appear associated with psychological distress and depression. Depression is one of the leading causes of disability and previous studies suggest that low-grade systemic inflammation may contribute to the development of depression. CRP is a commonly used marker of inflammation, and inflammatory disease is suspected when CRP levels are elevated.

Creatine (C₄H₉O₂N₃ or α-methyl guanidine-acetic acid) is a compound present in vertebrate muscle tissue, principally as phosphocreatine. Creatine is synthesized primarily in the liver and also in the pancreas and the kidneys. Creatine is eventually spontaneously degraded into creatinine by muscle and is released into the blood. It is then excreted by the kidneys and removed by the body by glomerular filtration. The amount of creatinine produced is relatively stable in a given person. Serum creatinine level is therefore determined by the rate it is being removed, which is roughly a measure of kidney function. If kidney function falls, serum creatinine level will rise. Thus, blood levels of creatinine are a good measure of renal function. Usually, increased creatinine levels do not appear unless significant renal impairment exists.

AChE is an enzyme that degrades, through hydrolytic activity, acetylcholine to produce choline and an acetate group. It is mainly found at neuromuscular junctions and cholinergic nervous system, where its activity serves to terminate synaptic transmission. The AChE enzyme has a very high catalytic activity, wherein each molecule being capable of degrading up to about 25,000 acetylcholine molecules per second. As used herein, the “AChE” encompasses all known and unknown isoforms of AChE and other enzymes with analogous activity including, but not limited to, butyrylcholinesterase (BuChE) unless the context clearly dictates otherwise.

AChE is a highly polymorphic enzyme, isoforms of which can be distinguished by their subunit associations and hydrodynamic properties. Differing sedimentation coefficients of different isoforms allow for their separation by ultracentrifugation on sucrose density gradients. In mammalian brain, the bulk of AChE occurs as a tetrameric, G4 form together with much smaller amounts of a monomeric, G1. There is strong evidence that not all AChE inhibitors inhibit all forms of AChE equally.

The G4 form of AChE is the major isoform in most regions within the brain. Approximately 60%-90% of this enzymatic form is extracellular. Extracellular G4 AChE is the major form for metabolizing acetylcholine (ACh) and this form is selectively depleted in Alzheimer's disease suggesting that G4 is the physiologically relevant isoform cholinergic synapses and its inhibition would be expected to prolong the action of AChE. By contrast, G1 occurs primarily in the neural cytoplasm where its inhibition would be unlikely to affect synaptic physiology, making G4 selective AChE inhibitors much more effective and potent.

The term “AChE inhibitor” means huperzine (including huperzine A, huperzine B, huperzine C), a huperzine analog (as defined below), or a non-huperzine AChE inhibitor, or their pharmaceutically accepted salts or solvates thereof, unless otherwise defined in a particular embodiment. AChE inhibitors may or may not have equal efficacy in different parts of the brain. In some embodiments of the present invention, the AChE inhibitor may be substantially equally effective in all regions of the brain. In some instances, AChE inhibitors inhibit AChE with similar mechanisms and to a similar degree. Yet, different AChE inhibitors effect on other cholinesterases such as, for example, BuChE, is specific to the particular compound being used.

“Huperzine A” is an AChE inhibitor with ring numbering shown:

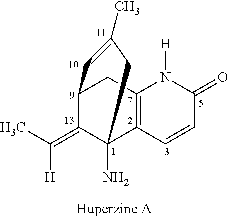

The term “huperzine” means huperzine A, huperzine B, or huperzine C, or their pharmaceutically accepted salts or solvates thereof, unless otherwise defined in a particular embodiment. Huperzine A is (1R,9,13E)-1-amino-13-ethylidene-11-methyl-6-azatricyclo[7.3.1.0^{2,7}]trideca-2(7),3,10-trien-5-one. Huperzine B is (4aR,5R,10bR)-2,3,4,4a,5,6-hexahydro-12-methyl-1H-5,10b-propeno-1,7-phenanthrolin-8(7H)-one, Huperzine C is (1R,9S, 13R)-1-amino-13-ethenyl-11-methyl-6-azatricyclo[7.3.1.0^{2,7}]trideca-2(7),3,10-trien-5-one.

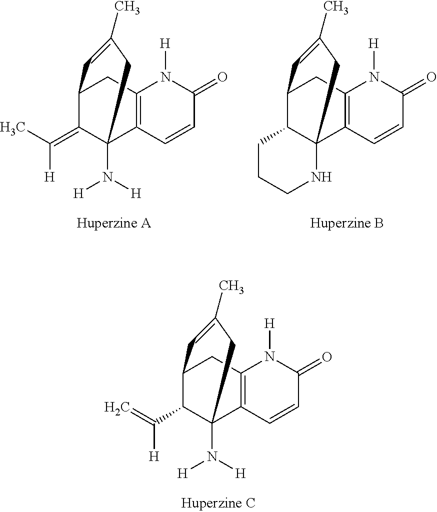 [SMILES: C/C=C1\[C@H]2C=C(C)C[C@]1(N)c1ccc(=O)[nH]c1C2 ; CC1=C[C@H]2Cc3[nH]c(=O)ccc3[C@]3(C1)NCCC[C@H]23 ; C=C[C@@H]1[C@H]2C=C(C)C[C@]1(N)c1ccc(=O)[nH]c1C2]

The term “huperzine analog” means a compound of general Formula 1 that is not huperzine:

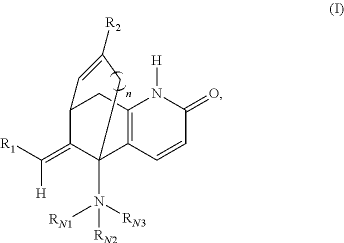

or pharmaceutically accepted salt or solvate,
wherein R₁ is selected from CH₃, CF₃, CF₂CF₃, CF₂CF₂CF3, SO₂CH₃, SO₂Ph, SO₂Ar, SO₃H, and SO₃Ar; R₂ is selected from an (C₁-C₂₄)alkyl, an aryl, a cycloalkyl, a (C₂-C₂₄)alkenyl, a heterocycle, and a heteroaryl; R_{N1} and R_{N2}, are independently selected from H, (C₁-C₂₄)alkyl, CF₃, CF₂CF₃, CCl₃, CBr₃, and CHO; R_{N3} is selected from absent and (C₁-C₂₄)alkyl; and n is an integer selected from 1, 2, 3, and 4; including Formulas II-VIII (as defined below).

The term “non-huperzine AChE inhibitor” means a compound that is a natural or synthetic compound that exhibits reversible or quasi-irreversible inhibition of AChE, but is not a huperzine or huperzine analog as defined above. Such compounds include, but are not limited to, carbamates, organophosphates, cannabinoids, phyostigmine, neostigmine, rivastigmine, pyridostigmine, ambenonium, demarcarium, tacrine, donepezil, distigmine, phenserine, galantamine, edrophonium, ladostigil, ungeremine, lactucopicrin, and their pharmaceutically acceptable salts and solvates, thereof.

The term “carbamate” means a non-huperzine AChE inhibitor that may include aldicarb, bendiocarb, bufencarb, carbaryl, carbendazim, carbetamide, carbofuran, carbosulfan, chlorbufam, choloropropham, ethiofencarb, formetanate, methiocarb, methomyl, oxamyl, phenmedipham, pinmicarb, pirimicarb, propamocarb, propham, and propoxur.

The term “organophosphate” means a non-huperzine AChE inhibitor that may include ecothiophate, diisopropl fluorophosphate, cadusafos, cyclosarin, dichlorvos, dimethoate, metrifonate, parathion, malathion, diazinon or their pharmaceutically accepted salt or solvate.

The term “cannabinoid” means a non-huperzine AChE inhibitor that may include Δ⁹-tetrahydrocannabinol, a synthetic cannabinoid, a semisynthetic cannabinoid, or their combination.

Embodiments are directed to a method of treating a seizure disorder by administering to a subject in need of such treatment a therapeutically effective amount of an AChE inhibitor, wherein the subject has an increased risk of a cardiac event from such seizure disorder, and wherein the AChE inhibitor decreases the risk of such cardiac event. In some embodiments, the method of treating a seizure disorder includes wherein the seizure disorder is one of epilepsy, Dravet Syndrome (Severe Myoclonic Epilepsy of Infancy, SMEI), generalized epilepsy with febrile seizures plus (GEFS+), and related disorders, and combinations thereof. In some embodiments, the risk of sudden unexplained death is decreased. In some embodiments, the method of treating a seizure disorder includes wherein the cardiac event is a heart attack, a stroke, cardiac arrest, an irregular heart rhythm, or tachycardia, or combinations thereof.

Embodiments are directed to a method of decreasing the e risk of a cardiac event in a subject with a seizure disorder by administering to the subject in need of such treatment a therapeutically effective amount of an AChE inhibitor, wherein the subject has an increased risk of a cardiac event from the seizure disorder, and wherein the AChE inhibitor decreases the risk of such cardiac event. In some embodiments, the method of decreasing the risk of a cardiac event in a subject with a seizure disorder includes wherein the seizure disorder is one of epilepsy. Dravet Syndrome (Severe Myoclonic Epilepsy of Infancy, SMEI), generalized epilepsy with febrile seizures plus (GEFS+), and related disorders, and combinations thereof. In some embodiments, the risk of sudden unexplained death is decreased. In some embodiments, the method of decreasing the risk of a cardiac event includes wherein the cardiac event is a heart attack, a stroke, cardiac arrest, an irregular heart rhythm, or tachycardia, or combinations thereof.

Embodiments are directed to a method of protecting the heart in a subject with a seizure disorder by administering to the subject in need of such treatment a therapeutically effective amount of an AChE inhibitor, wherein the subject has an increased risk of a heart damage from the seizure disorder, and wherein the AChE inhibitor decreases the risk of such heart damage. In some embodiments, the method of protecting the heart in a subject with a seizure disorder includes wherein the seizure disorder is one of epilepsy, Dravet Syndrome (Severe Myoclonic Epilepsy of Infancy, SMEI) generalized epilepsy with febrile seizures plus (GEFS+), and related disorders, and combinations thereof. In some embodiments, the risk of sudden unexplained death is decreased. In some embodiments, heart damage can be caused by a heart attack, a stroke, or cardiac arrest, or combinations thereof.

Embodiments are directed to a method of treating a seizure disorder by administering to a subject in need of such treatment a therapeutically effective amount of an AChE inhibitor, wherein the AChE inhibitor does not prolong the subject's QTc interval and wherein the seizure disorder is treated. In some embodiments, the seizure disorder is one of epilepsy, Dravet Syndrome (Severe Myoclonic Epilepsy of Infancy, SMEI), generalized epilepsy with febrile seizures plus (GEFS+), and related disorders, and combinations thereof. In some embodiments, the risk of sudden unexplained death is decreased.

Embodiments are directed to a method of decreasing the risk of a cardiac event in a subject without a seizure disorder by administering to the subject in need of such treatment a therapeutically effective amount of an AChE inhibitor, wherein the subject has an increased risk of a cardiac event and wherein the AChE inhibitor decreases the risk of such cardiac event. In some embodiments, the cardiac event is a heart attack, a stroke, cardiac arrest, an irregular heart rhythm, or tachycardia, or combinations thereof. In some embodiments, the risk of sudden unexplained death is decreased.

Embodiments are directed to a method of decreasing the risk of a cardiac event in a subject with electrocardiogram abnormalities by administering to the subject a therapeutically effective amount of an AChE inhibitor, wherein said subject has an increased risk of a cardiac event and wherein the ACME inhibitor decreases the risk of such cardiac event. In some embodiments, the cardiac event is a heart attack, a stroke, cardiac arrest, an irregular heart rhythm, or tachycardia, or combinations thereof. In some embodiments, the risk of sudden unexplained death is decreased.

Embodiments are directed to a method for treating a kidney disease by administering to a subject in need of such treatment a therapeutically effective amount of an AChE inhibitor, thereby treating the kidney disease. In some embodiments, the kidney disease is chronic kidney disease or acute kidney disease. In some embodiments, the kidney disease is chronic renal failure. In some embodiments, the chronic renal failure may be caused by progressive destruction of nephron mass, glomerulonephritis, tubulointerstitial diseases, diabetic nephropathy, or nephrosclerosis, or combinations thereof. In some embodiments, the kidney disease is renal dysfunction. In some embodiments, the kidney disease is acute renal failure. In some embodiments, the acute renal failure accompanies an acute kidney injury, a chronic kidney disease, acidosis, diabetic neuropathy, or acute-on-chronic failure, or a combination thereof. In some embodiments, the acute renal failure may include a rapid deterioration in renal function sufficient to result in accumulation of nitrogenous wastes in the body, renal hypoperfusion, obstructive uropathy, or intrinsic renal disease such as acute glomerulonephritis, or combinations thereof. In some embodiments, the kidney disease occurs with diabetic neuropathy. In some embodiments, the diabetic neuropathy includes complications of diabetes affecting nerves. In some embodiments, the diabetic neuropathy is a polyneuropathy, a focal mononeuropathy, a mononeuropathy affecting an oculomotor or abducens cranial nerve, or a multiple mononeuropathy, or a combination thereof. In some embodiments, the kidney disease is a drug induced diabetic neuropathy. In some embodiments, the drug induced diabetic neuropathy includes complications of diabetes affecting nerves which are caused by drugs, chemotoxicity, radiation, or a combination thereof. In some embodiments, the drug induced diabetic neuropathy may be a polyneuropathy, a focal mononeuropathy, a mononeuropathy affecting an oculomotor or abducens cranial nerve, or a multiple mononeuropathy, or a combination thereof. In some embodiments, the kidney disease is advanced chronic kidney disease in a type 2 diabetes patient. In some embodiments, the kidney disease is from acidosis. In some embodiments, the kidney disease is treated by improving kidney function, improving creatinine clearance, or improving glomerular filtration rates, or a combination thereof.

Embodiments are directed to a method of increasing or stabilizing creatinine clearance in a subject by administering to a subject in need of such treatment a therapeutically effective amount of an ACNE inhibitor, thereby increasing or stabilizing the creatinine clearance. In some embodiments, the need for such treatment may be due to a kidney disease as described in foregoing embodiments.

Embodiments are directed to a method of increasing or stabilizing glomerular filtration rate in a subject by administering to a subject in need of such treatment a therapeutic amount of an AChE inhibitor, thereby increasing or stabilizing the glomerular filtration rate. In some embodiments, the need for such treatment may be due to a kidney disease as described in foregoing embodiments.

Embodiments are directed to a method of reducing elevated CRP level in a subject by administering to the subject in need of such treatment a therapeutically effective amount of an AChE inhibitor, wherein the CRP level is reduced. In some embodiments, the elevated CRP level is greater than or equal to 5 mg/L, greater than 5 mg/L, greater than or equal to 7.5 mg/L, greater than 7.5 mg/L, greater than or equal to 10 mg/L, greater than 10 greater than or equal to 20 nag/L, greater than 20 or at a range between or including any two of these values. In some embodiments, the CRP level in a subject may be elevated above a normal baseline CRP level for that particular subject or for an average/normal subject. In some embodiments, the elevated CRP level may be caused by a kidney disease as described in foregoing embodiments. In some embodiments, the elevated CRP level is reduced by improving kidney function, improving creatinine clearance, or improving glomerular filtration rates, or a combination thereof. In some embodiments, the elevated CRP level is caused by a disease of the central nervous system, epilepsy, psychological distress, depression, a disease of the liver, acetaminophen toxicity, alcoholic liver disease, liver cirrhosis, primary liver cancer, liver cysts, liver fibrosis, non-alcoholic fatty liver disease, hepatitis, or primary sclerosing cholangitis, a disease of the heart, an atherosclerotic disease, atherosclerosis, coronary artery disease, cardiomyopathy, hypertensive heart disease, heart failure, endocarditis, stroke, stent-placement related restenosis, acute coronary syndrome, chronic kidney disease, rheumatoid arthritis, peripheral artery disease, chronic obstructive pulmonary disease, end stage renal disease, or systemic lupus erythrematosis, or a combination thereof.

Each of the foregoing embodiments includes the administration of a therapeutically effective amount of an AChE inhibitor, or a pharmaceutically acceptable salt or solvate thereof. In some embodiments, the AChE inhibitor is huperzine. In some embodiments, the AChE inhibitor is huperzine A, huperzine B, or huperzine C. In a preferred embodiment, the AChE inhibitor is huperzine A. In some embodiments, the AChE inhibitor is a huperzine analog, in some embodiments, the AChE inhibitor is a compound of Formula (I):

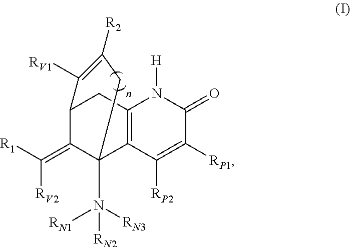

where R₁ is one of CH₃, CF₃, CF₂CF₃, CF₂CF₂CF₃, SO₂CH₃, SO₂Ph, SO₂Ar, SO₃H, and SO3Ar; R₂ is one of an (C₁-C₂₄)alkyl, an aryl, a cycloalkyl, a (C₂-C₂₄)alkenyl, a heterocycle, and a heteroaryl; R_{P1}, R_{P2}, R_{V1}, R_{V2} are each independently one of hydrogen and fluorine; R_{N1} and R_{N2} are each independently one of H, (C₁-C₂₄)alkyl, CF₃, CF₂CF₃, CCl₃, CBr₃, and CHO; R_{N3} is absent or a (C₁-C₂₄)alkyl; and n is the integer 1, 2, 3, or 4. In a preferred embodiment, R_{N1} and R_{N2} are independently one of a (C₁-C₂₄)alkyl, CF₃, CF₂CF₃, CCl₃, CBr₃, or CHO, and R_{N3} is a (C1-C₂₄)alkyl, In some embodiments, R_{N3} is absent and the 1-amino group is not a quaternary amine. In a preferred embodiment, R_{N3} is absent. In some embodiments, the quaternary amine has three independent alkyl groups. In some embodiments, the quaternary amine has three methyl groups. In some embodiments, the anionic counter ion of the quaternary amine is any pharmaceutically acceptable salt. In some embodiments, the pharmaceutically acceptable anionic counter ions are acetate, bromide, camsylate, chloride, formate, fumarate, maleate, mesylate, nitrate, oxalate, phosphate, sulfate, tartrate, thiocyanate, tosylate, adipate, caprate, caproate, caprylate, dodecylsulfate, glutarate, laurate, oleate, palmitate, sebacate, stearate, or undecylenate, or combinations thereof. In a preferred embodiment, the anionic counterions are acetate, bromide, camsylate, chloride, formate, fumarate, maleate, mesylate, nitrate, oxalate, phosphate, sulfate, tartrate, thiocyanate, or tosylate, or combinations thereof. In some embodiments, n is an integer selected from 2, 3, and 4. In a preferred embodiment, n is 2. In some embodiments, R₂ is phenyl and n is an integer selected from 2, 3, and 4. In some embodiments, R₂ is phenyl, R₁ is methyl, R_{N1} and R_{N2} are H, and R_{N3} is absent. In some embodiments, the AChE inhibitor is a huperzine analog compound of Formula II:

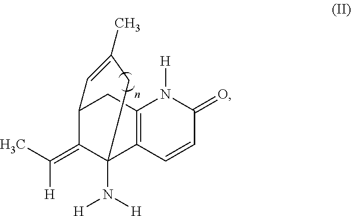

where n is selected from 2, 3, or 4. In a preferred embodiment, n is 2 with the proviso the compound is not huperzine A. In some embodiments, the AChE inhibitor is a huperzine analog compound of Formula III:

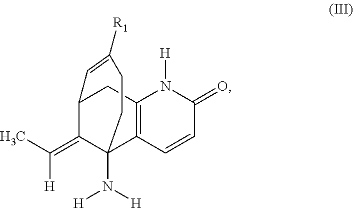

where R₁ is one of an (C₂-C₂₄)alkyl, an aryl, a cycloalkyl, (C₂-C₂₄)alkenyl, a heterocycle, or a heteroatyl. In a preferred embodiment, R₁ is a phenyl group. In some embodiments, the AChE inhibitor is a huperzine analog compound of Formula IV:

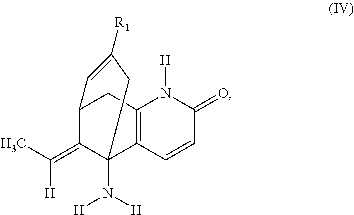

where R₁ is one of a (C₂-C₂₄)alkyl, an aryl, a cycloalkyl, a (C2-C₂₄)alkenyl, a heterocycle, or a Heteroaryl. In some embodiments, R₁ is a substituted phenyl group. In other embodiments, R₁ is a (C₂-C₂₀)alkyl. In a preferred embodiment, R₁ is a (C₂-C₂₄)alkyl. In a more preferred embodiment, R₁ is a (C₁-C₄)alkyl. In some embodiments, the alkyl may be substituted. In some embodiments, the AChE inhibitor is a huperzine analog compound of Formula V:

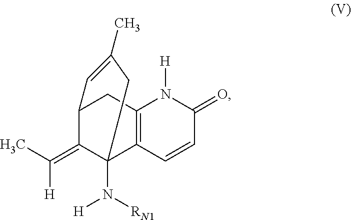

where R_{N1} is one of a (C₁-C₂₄)alkyl, CF₃, CF₂CF₃, CCl₃, CBr₃, CH₂OH, or CHO. In a preferred embodiment, R_{N1} is (C₁-C₂₀)alkyl. In a preferred embodiment, R_{N1} is (C₁-C₄)alkyl. In a preferred embodiment, R_{N1} is (C₁)alkyl. In a preferred embodiment, R_{N1} is (C₂-C₄)alkyl. In some embodiments, the alkyl may be substituted. In some embodiments, the AChE inhibitor is a huperzine analog compound of Formula VI:

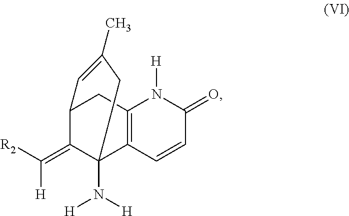

where R₂ is one of CF₂CF₃, CF₂CF₂CF₃, SO₂CH₃, SO₂Ph, SO₂Ar, SO₃H, or SO₃Ar. In some embodiments, the AChE inhibitor is a huperzine analog compound of Formula (VII):

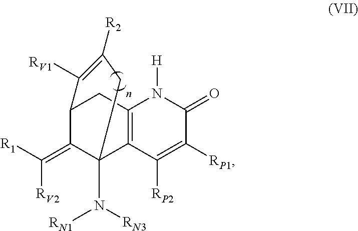

where R₁ is one of CH₃, CF₃, CF₂CF₃, CF₂CF₂CF₃, SO₂CH₃, SO₂Ph, SO₂Ar, SO₃H, or SO₃Ar; R₂ is one of a (C1-C₂₄)alkyl, an aryl, a cycloalkyl, (C₂-C₂₄)alkenyl, a heterocycle, and a heteroaryl; R_{P1}, R_{P2}, R_{V1}, R_{V2} are each independently H or F, but at least one of R_{P1}, R_{P2}, R_{V1}and R_{V2} is fluorine; R_{N1} and R_{N2} are independently one of H, (C₁-C₂₄)alkyl, CF₃, CF₂CF₃, CCl₃, CBr₃, or CHO; and n is selected from 1, 2, 3, or 4. In some embodiments, the AChE inhibitor is a huperzine analog compound of Formula VIII:

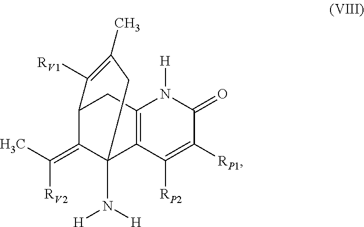

where R_{P1}, R_{P2}, R_{V1}, and R_{V2} are each independently H or F, but at least one of R_{P1}, R_{P2}, R_{V1}, and R_{V2} is fluorine.

In some embodiments, the AChE inhibitor is a non-huperzine AChE inhibitor. In some embodiments, the non-huperzine AChE inhibitor exhibits reversible or quasi-irreversible inhibition of AChE. In some embodiments, the AChE inhibitor is a carbamate, un organophosphate, a cannabinoid, phyostigmine, neostigmine, rivastigmine, pyridostigmine, ambenonium, demarcarium, tacrine, donepezil, distigmine, phenserine, galantamine, edrophonium, ladostigil, ungeremine, or lactucopicrin. In a preferred embodiment, the AChE inhibitor is donepezil. In some embodiments, the AChE inhibitor is aldicarb, bendiocarb, bufencarb, carbaryl, carbendazim, carbetamide, carbofuran, carbosulfan, chlorbufuran, choloropropham, ethiofencarb, formetanate, methiocarb, methomyl, oxamyl, phenmedipham, pinmicarb, pirimicarb, propamocarb, propham, propoxur. In some embodiments, the AChE inhibitor is ecothiophate, diisopropyl fluorophosphate, cadusafos, cyclosarin, dichlorvos, dimethoate, metrifonate, parathion, malathion, diazinon. In some embodiments, the AChE inhibitor is Δ⁹-tetrahydrocannabinol, a synthetic cannabinoid, or a semisynthetic cannabinoid.

In some embodiments, a combination of AChE inhibitors is administered.

In some embodiments, the AChE inhibitor is administered without the administration of a non-steroidal anti-inflammatory drug (NSAID).

In some embodiments, the therapeutically effective dose of the AChE inhibitor is 0.4 mg/day to 1500 mg/day, 0.8 mg/day to 6.4 mg/day, preferably 1.2 mg/day to 3.2 mg/day, 1.6 mg/day to 2.4 mg/day, or 2.5 mg/day to 10 mg/day, or any range between or including any two of these values. In a preferred embodiment, the therapeutically effective dose is 2.5 mg/day, to 10 mg/day. In some embodiments, the therapeutically effective dose is 0.4 mg/day, 0.6 mg/day, 0.8 mg/day, 1.2 mg/day, 1.6 mg/day, 2.0 mg/day, 2.4 mg/day, 2.8 mg/day, 3.2 mg/day, 3.6 mg/day, 4.0 mg/day, or 6.4 mg/day, or any range between or including any two of these values. In some embodiments, the therapeutically effective dose is 0.01 mg/kg/day to 20 mg/kg/day. In some embodiments, the therapeutically effective dose is 1 mcg/kg, 2 mcg/kg, 5 mcg/kg, 10 mcg/kg, 20 mcg/kg, 30 mcg/kg 60 mcg/kg, 120 mcg/kg, 240 mcg/kg, 500 mcg/kg, or 1 mg/kg, or any range between or including any two of these values. In some embodiments, the AChE inhibitor may be dosed daily, twice daily, three times daily, four times daily, five times daily, six times daily, or eight times daily.

The amount of AChE inhibitor to be administered is that amount which is therapeutically effective. The dosage to be administered and dosage regimen will depend on the characteristics of the subject being treated, e.g., the particular animal treated, age, weight, health, types of concurrent treatment, if any, and frequency of treatments, and can be easily determined by one of skill in the art (e.g., by the physician/clinician). The dosage regimen is to be adjusted or titrated by the physician/clinician according to methods known to the physician/clinician in order to obtain the optimal clinical response.

Specific modes of administration will depend on the indication. The selection of the specific route of administration will depend on the characteristics of the subject being treated, e.g., the particular animal treated, age, weight, health, types of concurrent treatment, if any, and frequency of treatments, and can be easily determined by one of skill in the art (e.g., by the physician/clinician).

The compounds and compositions of AChE inhibitors of all aspects of the methods of the present invention can be administered in the conventional manner by any route where they are active. In some embodiments, administration can be systemic, topical, or oral. In some embodiments, for example, administration can be, but is not limited to, parenteral, subcutaneous, intravenous, intramuscular, intraperitoneal, intraarterial, intraadipose, intraarticular, intrathecal, sublingual, intranasal, rectal, transdermal, oral, buccal, or ocular routes, or intravaginally, by inhalation, by depot injections, by implants, or by local delivery by catheter or stent. In some embodiments, administration is via a dosage form other than an immediate release dosage form. In some embodiments, administration is via a slow release dosage form, an extended release dosage form, or a sustained release dosage form, or a combination thereof. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

In some embodiments, pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. In some embodiments, the active ingredients can be contained in such formulations with pharmaceutically acceptable diluents, fillers disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

In some embodiments, the compounds of the present invention can be formulated for parenteral administration by injection, e.g., by bolus injection or continuous infusion. In some embodiments, the compounds can be administered by continuous infusion subcutaneously over a period of about 15 minutes to about 24 hours. In some embodiments, formulations for injection can be presented in unit dosage form, e.g., in ampoules or in multi-dose containers, with an added preservative. In some embodiments, the compositions can take such forms as suspensions, solutions or emulsions in oily or aqueous vehicles, and can contain formulatory agents such as suspending, stabilizing and/or dispersing agents.

In some embodiments, for oral administration, the compounds can be formulated readily by combining these compound with pharmaceutically acceptable carriers well known in the art. In some embodiments, such carriers enable the compounds of the invention to be formulated as tablets, pills, dragees, gum dragees, capsules, liquids, gels, syrups, slurries, suspensions, powders, and the like, for oral ingestion by a patient to be treated. In some embodiments, pharmaceutical preparations for oral use can be obtained by adding a solid excipient, optionally grinding the resulting mixture, and processing the mixture of granules, after adding suitable auxiliaries, if desired, to obtain tablets or dragee cores. In some embodiments, suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

In some embodiments, dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. In some embodiments, dyestuffs or pigments can be added to the tablets or dragee coatings for identification or to characterize different combinations of active compound doses.

In some embodiments, pharmaceutical preparations which can be used orally include, but are not limited to, push-fit capsules made of gelatin, as well as soft, sealed capsules made of gelatin and a plasticizer, such as glycerol or sorbitol. In some embodiments, the push-fit capsules contain the active ingredients in admixture with filler such as, e.g., lactose, binders, such as, e.g., starches, and/or lubricants such as, e.g., talc or magnesium stearate and, optionally, stabilizers. In some embodiments, in soft capsules, the active compounds are dissolved or suspended in suitable liquids, such as fatty oils, liquid paraffin, or liquid polyethylene glycols. In some embodiments, stabilizers are added. All formulations for oral administration should be in dosages suitable for such administration.

In some embodiments, for buccal administration, the compositions take the form of, e.g., tablets or lozenges formulated in a conventional manner.

In some embodiments, for administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In some embodiments, the compounds of the present invention are formulated in rectal compositions such as suppositories or retention enemas, e.g., containing conventional suppository bases such as cocoa butter or other glycerides.

In addition to the formulations described previously, in some embodiments, the compounds of the present invention are formulated as a depot preparation. Such long acting formulations can be administered by implantation (for example subcutaneously or muscularly) or by intramuscular injection.

In some embodiments, depot injections are administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In some embodiments, in transdermal administration, the compounds of the present invention are applied to a plaster, or can be applied by transdermal, therapeutic systems that are consequently supplied to the organism.

In some embodiments, pharmaceutical compositions of the compounds comprise suitable solid or gel phase carriers or excipients. In some embodiments, such carriers or excipients include, but are not limited to, calcium carbonate, calcium phosphate, various sugars, starches, cellulose derivatives, gelatin, and polymers such as, e.g., polyethylene glycols.

In some embodiments, the compounds of the present invention are administered in combination with other active ingredients, such as, for example, adjuvants, protease inhibitors, or other compatible (hugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

The present disclosure should not be considered limited to the particular embodiments described above, but rather should be understood to cover all aspects of the disclosure as fairly set out in the attached claims. Various modifications as well as numerous structures to which the present disclosure may be applicable, will be readily apparent to those skilled in the art to which the present disclosure is directed upon review of the present specification. The claims are intended to cover such modifications and devices, The invention and embodiments thereof illustrating the method and materials used may be further understood by reference to the following non-limiting examples.

### EXAMPLE 1

A phase 1 clinical trial was conducted with huperzine A. The primary aim of the study was to conduct a proof-of-principle assessment of the safety and tolerability and early stage pharmacokinetics of dietary supplement Huperzine A up to 6.4 mg/day as add-on, open-label therapy in 8 subjects with drug-resistant epilepsy. The hypothesis was that Huperzine A in 8 subjects with drug-resistant epilepsy as add-on; open-label therapy would be well tolerated when titrated from 1.6 mg/day up to 6.4 mg/day. The secondary aim was to acquire preliminary data on the parasympathetic effect of Huperzine A on cardiac function.

The trial was a single-center, in-patient, open-label, dose-escalation study conducted at one site. This study would enroll up to 10 adults (≧18 years to 65 years) to obtain 8 randomized subjects with drug-resistant epilepsy that were not well controlled with 1 to 3 concomitant anti-epileptic drugs (AEDs).

Inclusion criteria included:
1. Be males or females between 18-65 years of age;
2. Have a diagnosis of drug-resistant epilepsy by an epileptologist;
3. Have had a minimum of 3 seizures per month for the two months preceding enrollment into the study;
4. Be receiving stable doses (for at least 4 weeks) of one to three currently marketed AEDs, with or without vagus nerve stimulation (in which case the subject should be on the same stimulation parameters for at least 4 weeks);
5. Be in good general health, other than having epilepsy, in the judgment of the Principal Investigator based upon medical history, physical examination, standard 12-lead ECG, and clinical laboratory evaluations obtained within the two weeks prior to enrollment; and
6. Have had a brain MRI/CT within 10 years prior to enrollment showing no evidence of a neurological condition likely to progress. Conditions leading to exclusion include: brain tumor, active encephalitis, active meningitis or abscess.

A participant was ineligible to enter the study he/she meets one or more of following criteria:
1. Has taken Huperzine A;
2. Has ongoing nonepileptic events that could be confused by the subject and/or study staff as epileptic seizures or a history of such non-epileptic events within the last 2 years;
3. Has seizures that are uncountable; for example, they occur in clusters;
4. Has a pre-existing medical condition (including an existing progressive or degenerative neurological disorder) or takes medications that, in the Principal investigator's opinion, could interfere with the subject's suitability for participation in the study;
5. Has a history or evidence of significant psychiatric disturbance or illness, including alcohol or drug abuse within the past 2 years, or symptoms of psychosis (hallucinations, delusions) in the last 5 years;
6. Has a history of status epilepticus in the 12 months prior to enrollment.
7. Has had any clinical laboratory abnormalities within the past two months, prior to screening, considered of clinical significance by the Principal Investigator;
8. is on concomitant therapy with non-AED drugs that are cholinergic or that are active against the NMDA receptor; and
9. is currently taking or has taken Epigallocatechin gallate (EGCG) or green tea in the past 30 days.

The study drug was supplied as 0.200 mg tablets and dispensed by the study site's pharmacy. Huperzine A tablets derived from Huperzia serrata were sourced from a commercially available supplier in the United States.

Randomly chosen tablets of the supplied trial medication, as well as a composite have been assessed for purity by an independent laboratory using standard analytic techniques (HPLC) and found to contain between 0.145 mg and 0.166 mg of Huperzine A per tablet. Subject dosing was based on the label potency of the tablets (i.e. 0.200 mg). Tablets were randomly assigned to each subject in a dosing bottle container containing 120 tablets per subject. Each bottle was labeled with a study identifier. subject ID, dose of each tablet, dosing instructions, expiration date, and lot number.

Eight subjects with drug-resistant epilepsy were provided huperzine A. Six subjects completed the study. Doses were varied as in Table A. Doses were six hours apart. Subjects 1-3 began dosing at 18:00, subjects 4-8 began dosing at 12:00. Peak plasma level of huperzine A is reported in Table A.

TABLE A
Dosage of Subjects with Huperzine A

| | Subject: | | | | | | | |
|---|---|---|---|---|---|---|---|---|
| Dose: | 1 | 2 | 3 | 4 | 5 | 6 | 7 | 8 |
| 1 | 0.40 mg | 0.40 mg | 0.40 mg | 0.40 mg | 0.40 mg | 0.40 mg | 0.40 mg | 0.40 mg |
| 2 | 0.80 mg | 0.80 mg | 0.40 mg | 0.40 mg | 0.40 mg | 0.40 mg | 0.40 mg | 0.40 mg |
| 3 | 0.80 mg | 0.80 mg | 0.80 mg | 0.40 mg | 0.40 mg | 0.40 mg | 0.40 mg | 0.40 mg |
| 4 | 0.40 mg | | 0.80 mg | 0.40 mg | 0.40 mg | 0.40 mg | 0.40 mg | 0.40 mg |
| 5 | | | 0.60 mg | 0.60 mg | 0.60 mg | 0.60 mg | 0.60 mg | 0.60 mg |
| 6 | | | 0.60 mg | 0.60 mg | 0.60 mg | 0.60 mg | 0.60 mg | 0.60 mg |
| 7 | | | 0.40 mg | 0.60 mg | 0.60 mg | 0.60 mg | 0.60 mg | 0.60 mg |
| 8 | | | 0.40 mg | 0.60 mg | 0.60 mg | 0.60 mg | 0.60 mg | 0.60 mg |
| 9 | | | 0.40 mg | 0.40 mg | 0.80 mg | 0.60 mg | 0.60 mg | 0.60 mg |
| 10 | | | 0.40 mg | 0.40 mg | 0.80 mg | 0.60 mg | 0.60 mg | 0.60 mg |
| 11 | | | 0.40 mg | 0.40 mg | 0.60 mg | 0.40 mg | 0.40 mg | 0.40 mg |
| 12 | | | 0.40 mg | | 0.40 mg | 0.40 mg | 0.40 mg | 0.40 mg |
| 13 | | | | | 0.40 mg | 0.40 mg | 0.40 mg | 0.40 mg |
| Peak Plasma | 2.6 | 6.7 | 9.6 | 11.8 | 12.9 | 15.9 | 13.2 | TBD |
| ng/mL: | | | | | | | | |

Table B provides biomarker test results. Baseline, peak and change values represented as mean±the standard error of the mean (SEM). Statistical data is included for all subjects excluding subject #7 because that subject was on a conflicting medication. Peak represents peak plasma level or, if unavailable, the nearest values for all values except cardiological markers where peak represents Day 2. The p-values were calculated from one-sided paired t tests.

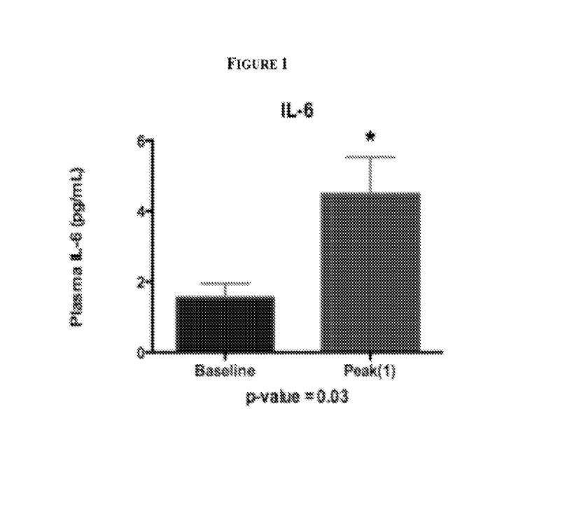

Inflammatory markers were tested on subjects during the clinical trial. Peak plasma levels of IL-6 were reported (Table B, FIG. 1). A statistically significant increase in IL-6 vas observed in subjects. The IL-6 increased 115% from an average of 1.3±0.2 pg/mL to 2.8±0.7 pg/mL (p-value 0.03). Testing for IL-6 using a different test center indicated a 133% increase in IL-6. An increase in IL-6 has positive clinical significance. Controlled increases in IL-6 are neuroprotective and anticonvulsant. inflammatory markers IL-1 beta, IL-10, and IL-17 showed lesser increases during the clinical trial.

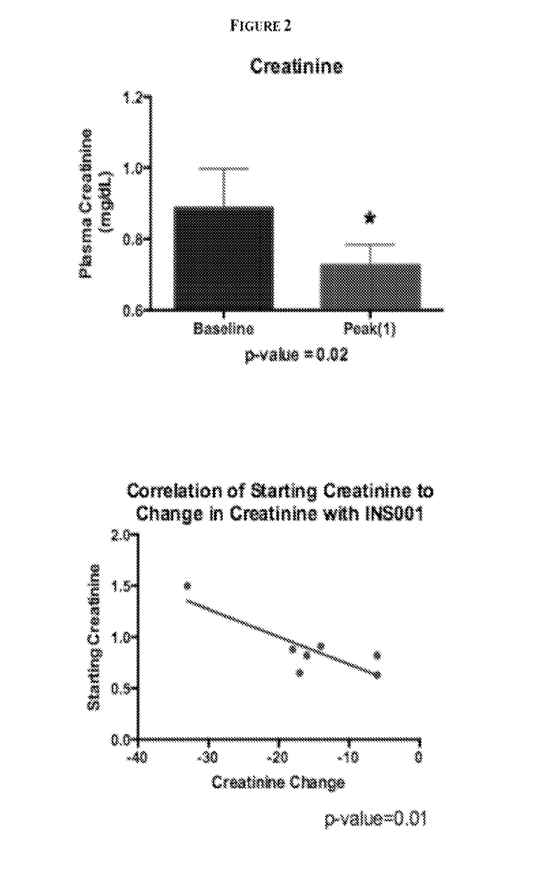

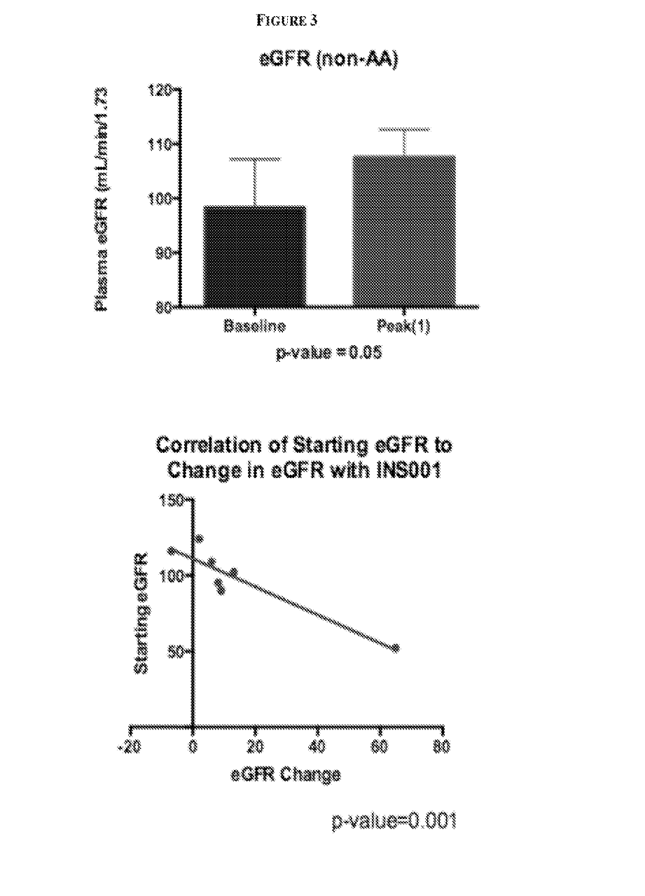

Data from subjects points o a statistically significant decrease in serum creatinine (FIG. 2). Serum creatinine decreased 18% from an average of 0.9+0.3 mg/dL (p-value=0.02), with a negative correlation of starting creatinine to a change in creatinine (p-value=0.01). A statistically significant increase in eGFR (creatinine clearance rates) was observed (FIG. 3). Creatinine clearance rates increased 9% from an average of 98.3±23.5 mL/min/1.73 (p-value=0.05), with a negative correlation of starting eGFR to a change in eGFR (p-value=0.001).

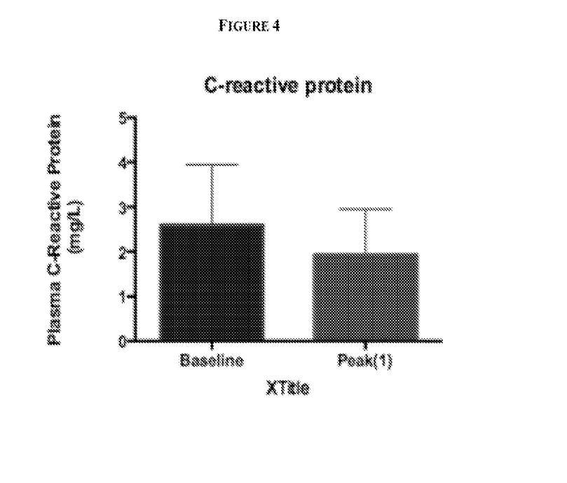

Data from subjects points to a statistically significant decrease in CRP (FIG. 4). CRP decreased 29% from an average of 2.6±3.0 mg/L (p-value 0.02). Cardiovascular benefits may provide a benefit unique to the epilepsy subject population and may have protective effects against sudden unexpected death in epilepsy. The improvements in biomarkers provide subjects with chronic kidney disease may benefit as most subjects with chronic kidney disease die of related cardiovascular disease.

Monocyte chemotactic protein-1 (MCP-1) as a marker is implicated in pathogeneses of several diseases characterized by monocytic infiltrates. MCP-1 is involved in the neuroinflammatory processes that take place in the various diseases of the central nervous system, which are characterized by neuronal degeneration. MCP-1 expression in glial cells is increased in epilepsy, brain ischemia, Alzheimer's disease, some encephalomyelitis, and traumatic brain injury. Levels of MCP-1 decreased 15% from an average of 175.2±43.5 to 148.9.±-26.3 pg/mL (Table B).

TABLE B
Dosage of Subjects with Huperzine A

| | | | | | p-value | p-value |
|---|---|---|---|---|---|---|
| | | Peak | | % | w/o #7 | with #7 |
| Units | Baseline | (1) | Change | Change | (2) | (2) |

Cardiologic Markers

| | | | | | | | |
|---|---|---|---|---|---|---|---|
| CRP | mg/L | 2.6 ± 3.0 | 1.8 ± 2.3 | −0.7 ± 0.8 | −29% | 0.02 | 0.26 |

Renal Markers

| | | | | | | | |
|---|---|---|---|---|---|---|---|
| Serum creatine | mg/dL | 0.9 ± 0.3 | 0.7 ± 0.1 | −0.2 ± 0.2 | −18% | 0.02 | 0.02 |
| eGFR | mL/min/ | 98.3 ± 23.5 | 107.6 ± 13.4 | 9.3 ± 12.7 | 9% | 0.05 | 0.05 |
| (flow rate) | 1.73 | | | | | | |

Inflammatory Markers

| | | | | | | | |
|---|---|---|---|---|---|---|---|
| IL-1 beta | pg/mL | 23.9 ± 12.4 | 30.0 ± 9.8 | 6.1 ± 5.2 | 26% | 0.14 | 0.11 |
| IL-6 (IITR1) | pg/mL | 1.3 ± 0.2 | 2.8 ± 0.7 | 1.5 ± 1.5 | 115% | 0.03 | 0.04 |
| IL-6 (Labcorp) | pg/mL | 1.8 ± 1.2 | 4.2 ± 2.4 | 2.4 ± 2.6 | 133% | 0.03 | 0.05 |
| IL-10 | pg/mL | 7.3 ± 2.7 | 8.8 ± 1.5 | 1.6 ± 1.6 | 21% | 0.21 | 0.11 |
| IL-17 | pg/mL | 35.1 ± 17.7 | 36.1 ± 19.2 | 1.0 ± 1.0 | 3% | 0.33 | 0.33 |
| TNF-alpha | pg/mL | 1.6 ± 0.4 | 1.3 ± 0.4 | −0.2 ± 0.1 | −15% | 0.005 | 0.005 |

Other markers

| | | | | | | | |
|---|---|---|---|---|---|---|---|
| MCP-1 | pg/mL | 175.2 ± 43.5 | 148.9 ± 26.3 | −26.3 ± 28.6 | −15% | 0.20 | 0.29 |

Baseline, peak and change values represented as mean ± SEM; data for all subjects excluding subject #7 (conflicting medication).
(1) Peak represents peak plasma level or, if unavailable, the nearest values for all values except cardiological markers where peak represents Day 2.
(2) p-values calculated from one-sided paired t tests.

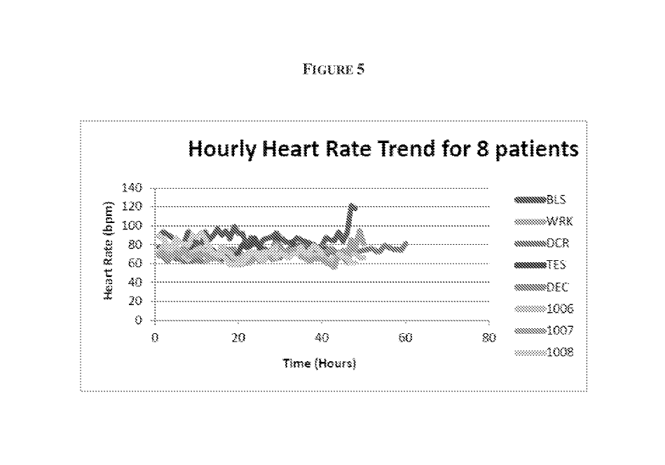

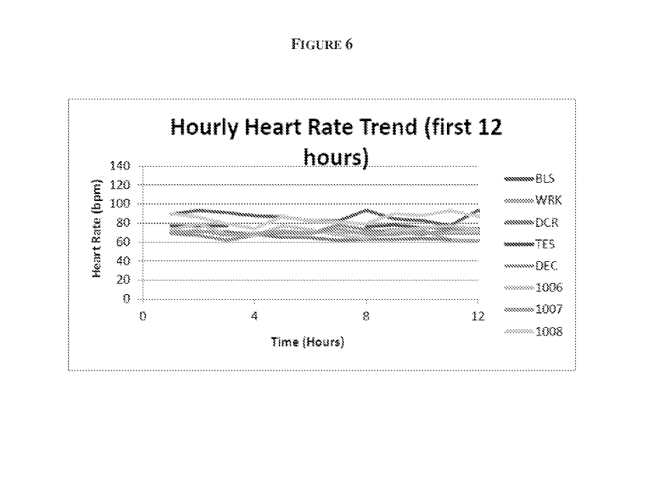

Heart rate, heart rate variability. T-wave alternans, QT interval, ventricular premature beats, and ventricular tachycardia were studied in all 8 subjects enrolled in the study. Heart rate was stable throughout the recordings in all but one subject. This individual experienced transient peaks in heart rate and a few ventricular premature beats, probably due to an emesis-related surge in sympathetic nerve activity. FIG. 5 shows hourly heart rate trends for the 8 enrolled subjects for the full recording period. FIG. 5 shows that only subject BLS exhibited an increase in heart rate to about 120 beats/min, which was apparently related to emesis. ECG recordings show a transient disconnect in subjects WRK and DEC. FIG. 6 shows hourly heart rate trends for 8 subjects for the initial 12 hours of recording.

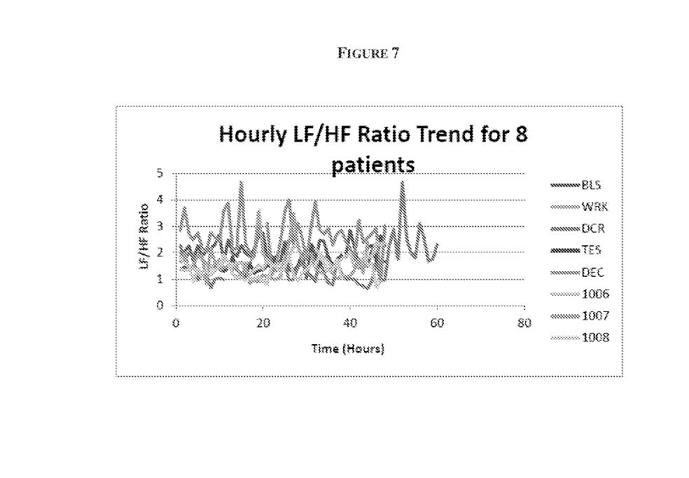

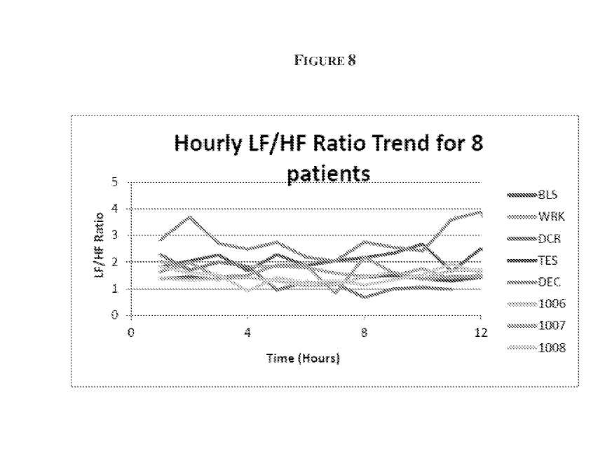

With regard to heart rate variability, the stable low frequency/high frequency (LF/HF) ratio data, a marker of sympathetic modulation, is consistent with the vagomimetic action of huperzine. FIG. 7 shows hourly heart rate variability trends for 8 subjects for the full recording period. A LF/HF ratio from about 1 to about 4 is in the normal range. FIG. 8 shows hourly heart rate variability trends for 8 subjects for the initial 12 hours of recording. During the first 6 hours, there is a trend toward lowering of the LF/HF ratio, which suggests a shift toward vagal dominance.

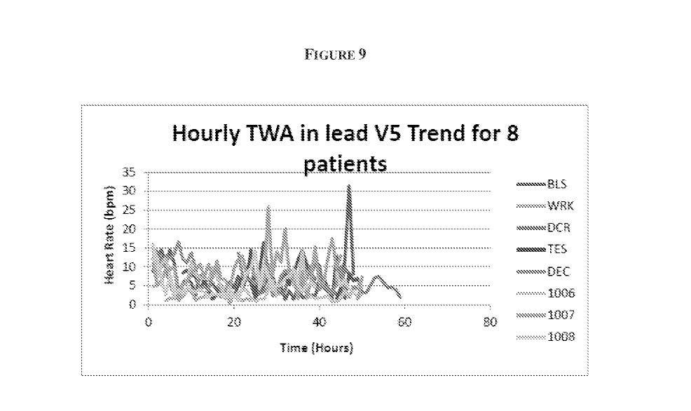

T-wave alternans (TWA) was generally low in all leads. FIG. 9 shows hourly trends in TWA for the 8 subjects for the full recording period. TWA became elevated to about 25 μV in subject 1007 and to about 30 μV during the final 120-beat/mm heart rate surge in subject BLS, however these TWA levels are in the normal range. The T-wave is a particular subportion of the electrocardiogram which is susceptible to pathologic alteration by repeated seizures, more specifically the pathology is abnormal variations in TWA. Abnormal excursions in TWA are well known to increase risk for fatal cardiac rhythms such as ventricular tachycardias. It was a surprising finding that the huperzines and huperzine analogs decrease abnormal excursions of TWA, particularly in people who have a history of uncontrolled epilepsy. In fact, another novel and surprise finding was that the huperzines and huperzine analogs act to normalize abnormal heart rhythms that could lead to fatal rhythms, such as those associated with TWA. It is hypothesized that the huperzines and huperzine analogs exert this beneficial effect on TWA by causing an increase in the available acetylcholine by inhibiting AChE. Acetylcholine then acts to slow cardiac contractions. Because the huperzines and huperzine analogs slow cardiac contractions, it would be likely that the huperzines and huperzine analogs would cause detrimental bradycardia. However, it was also a surprising finding that the huperzines and huperzine analogs provided beneficial effects on TWA, but no clinically significant degree of bradycardia.

The surprising finding that the huperzines and huperzine analogs act to normalize abnormal heart rhythms which can lead to fatal rhythms, such as those associated with TWA, indicates that the huperzines and huperzine analogs may also provide cardioprotection for other groups of patients without epilepsy who exhibit abnormal heart waves, such as abnormal TWA, due to known or unknown causes.

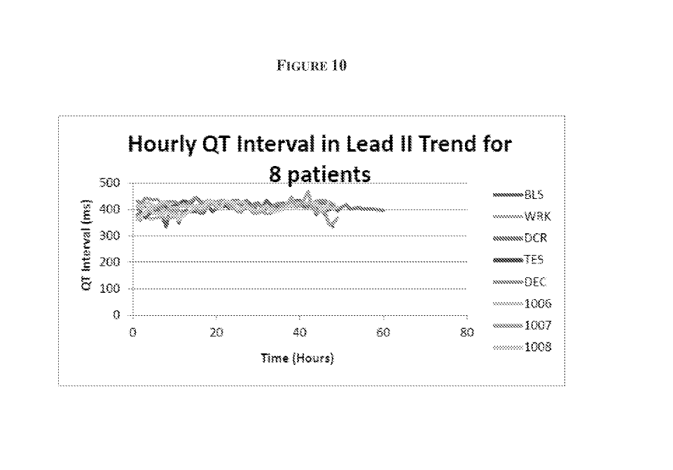

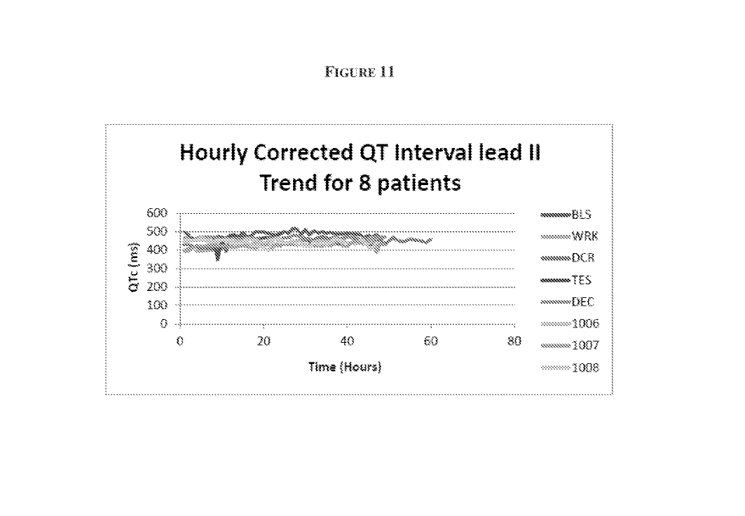

A study of the QM interval, a marker of repolarization, was unaltered. This is consistent with the absence of arrhythmia. FIG. 10 shows hourly trends in QT interval length for the 8 subjects for the full recording period. FIG. 11 shows hourly trends in QTc interval length for the 8 subjects for the full recording period as corrected using Bazett's formula.

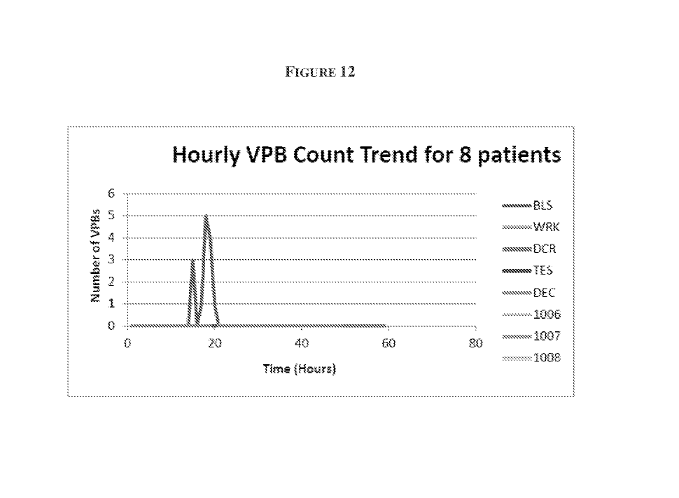

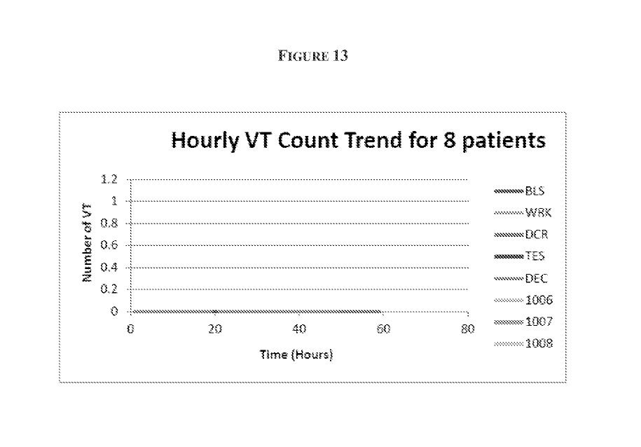

The ventricular premature beat (VPB) count is low and no episodes of ventricular tachycardia occurred in any of the 8 subjects. FIG. 12 shows hourly trends in VPB counts for the 8 subjects for the full recording period. Only one subject (BLS) exhibited instances of 3 to 5 ventricular premature beats, which are likely related to periods of generalized increases in heart rate. FIG. 13 shows that none of the 8 subjects exhibited ventricular tachycardia during the entire recording period.

Overall, none of the 8 subjects in the study exhibited an increase in QTc interval or had any episodes of ventricular tachycardia. 7 of the 8 subjects exhibited stable heart rates and no ventricular premature beats. The single exception experienced episodes of emesis, which was associated with 3 to 5 isolated ventricular premature beats. It can be concluded that cardiac electrical activity remains normal after huperzine administration. The results also indicate that huperzine may be vagomimetic, as assessed by heart rate variability. The subjects also exhibited low TWA levels, which is indicative cardiac electrical stability.

Cardiovascular benefits may provide a benefit unique to the epilepsy patient population and may have protective effects against sudden unexpected death in epilepsy. The improvements in biomarkers demonstrated in subjects with chronic kidney disease may benefit patients with chronic kidney disease as most die of related cardiovascular disease.

Although the present invention has been described in considerable detail with reference to certain preferred embodiments thereof, other versions are possible. Therefore, the spirit and scope of the appended claims should not be limited to the description and the preferred versions contained within this specification.

What is claimed is:

1. A method of treating a seizure disorder comprising administering to a subject in need of such treatment a therapeutically effective amount of an acetylcholinesterase (AChE) inhibitor, wherein the subject has an increased risk of a cardiac event from such seizure disorder, and wherein the AChE inhibitor decreases the risk of such cardiac event.

2. The method of claim 1, wherein the seizure disorder is selected from epilepsy, Dravet Syndrome (Severe Myoclonic Epilepsy of Infancy, SMEI), generalized epilepsy with febrile seizures plus (GEFS+), and related disorders, and combinations thereof.

3. The method of claim 2, wherein the risk of sudden unexplained death is decreased.

4. The method of claim 1, wherein the cardiac event is selected from a heart attack, a stroke, cardiac arrest, an irregular heart rhythm, and tachycardia, and combinations thereof.

5. The method of claim 1, wherein the AChE inhibitor is a compound of formula I: 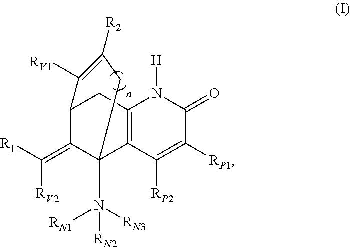
a pharmaceutically acceptable salt thereof, or a pharmaceutically acceptable solvate thereof,
wherein R₁ is selected from CH₃, CF₃, CF₂CF₃, CF₂CF₂CF₃, SO₂CH₃, SO₂Ph, SO₂Ar, SO3H, and SO₃Ar;
R2 is selected from an (C₁-C₂₄)alkyl, an aryl, a cycloalkyl, a (C₂-C₂₄)alkenyl, a heterocycle, and a heteroaryl;
R_{P1}, R_{P2}, R_{V1}, R_{V2} are independently selected from hydrogen and fluorine;
R_{N1} and R_{N2} are independently selected from H, (C₁-C₂₄)alkyl, CF₃, CF₂CF₃, CCl₃₅ CBr₃, and CHO;
R_{N3} is selected from absent and (C₁-C₂₄)alkyl; and
n is an integer selected from 1, 2, 3, and 4.

6. The method of claim 1, wherein the AChE inhibitor is selected from the group consisting of huperzine A, huperzine B, and huperzine C, and salts and solvates thereof, sand combinations thereof.

7. The method of claim 1, wherein the AChE inhibitor is huperzine A.

8. The method of claim , wherein the AChE inhibitor is administered to the subject at a dose selected from 0.8 mg/day to 6.4 mg/day, 1.2 mg/day to 3.2 mg/day, 1.6 mg/day to 2.4 mg/day, 0.01 mg/kg/day to 20 mg/kg/day, 0.5 mg/day to 1500 mg/day, and 2.5 mg/day to 10 mg/day.

9. A method of decreasing the risk of a cardiac event in a subject with a seizure disorder comprising administering to the subject in need of such treatment a therapeutically effective amount of an acetylcholinesterase (AChE) inhibitor, wherein said subject has an increased risk of a cardiac event from said seizure disorder, and wherein the ACME inhibitor decreases the risk of such cardiac event.

10. The method of claim 9, wherein the seizure disorder is selected from epilepsy, Dravet Syndrome (Severe Myoclonic Epilepsy of Infancy, SMEI), generalized epilepsy with febrile seizures plus (GEFS+), and related disorders, and combinations thereof.

11. The method of claim 10, wherein the risk of sudden unexplained death is decreased.

12. The method of claim 9, wherein the cardiac event is selected from a heart attack, a stroke, cardiac arrest, an irregular heart rhythm, and tachycardia, and combinations thereof.

13. The method of claim 9, wherein the AChE in inhibitor is a compound of Formula I: 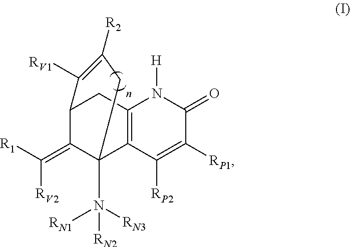
a pharmaceutically acceptable salt thereof, or a pharmaceutically acceptable solvate thereof,
wherein R₁ is selected from CH₃, CF₃, CF₂CF3, CF₂CF₂CF₃, SO₂CH₃, SO₂Ph, SO₂Ar, SO3H, and SO₃Ar;
R₂ is selected from an (C₁-C₂₄)alkyl, an aryl, a cycloalkyl, a (C₂-C₂₄)alkenyl, heterocycle, and a heteroaryl;
R_{P1}, R_{P2}, R_{V1}, R_{V2} are independently selected from hydrogen and fluorine;
R_{N1} and R_{N2} are independently selected from H, (C₁-C₂₄)alkyl, CF₃, CF2CF₃, CCl₃, CBr₃, and CHO;
R_{N3} is selected from absent and (C₁-C₂₄)alkyl; and
n is an integer selected from 1, 2, 3, and 4.

14. The method of claim 9, wherein the AChE inhibitor is selected from the group consisting of huperzine A, huperzine B, and huperzine C, and salts and solvates thereof, and combinations thereof.

15. The method of claim 9, wherein the AChE inhibitor is huperzine A.

16. The method of claim 9, wherein the AChE inhibitor is administered to the subject at a dose selected from 0.8 mg/day to 6.4 mg/day, 1.2 mg/day to 3.2 mg/day, 1.6 mg/day to 2.4 mg/day, 0.01 mg/kg/day to 20 mg/kg/day, 0.5 mg/day to 1500 mg/day, and 2.5 mg/day ⁻to 10 mg/day.

17. A method of treating a seizure disorder comprising administering to a subject in need of such treatment a therapeutically effective amount of an acetylcholinesterase (AChE) inhibitor, wherein the AChE inhibitor does not prolong said subject's QTc interval and wherein the seizure disorder is treated.

18. A method of decreasing the risk of a cardiac event a subject without a seizure disorder comprising administering to the subject in need of such treatment a therapeutically effective amount of an acetylcholinesterase (AChE) inhibitor, wherein said subject has an increased risk of a cardiac event and wherein the AChE inhibitor decreases the risk of such cardiac event.

19. A method for treating a kidney disease comprising administering to a subject in need of such treatment a therapeutically effective amount of an acetylcholinesterase (AChE) inhibitor, wherein the kidney disease is treated.

20. A method of reducing an elevated C-reactive protein (CRP) level in a subject comprising administering to a subject in need of such treatment a therapeutically effective amount of an acetylcholinesterase (AChE) inhibitor, wherein the CRP level is reduced.
